(19) United States
(12) Patent Application Publication
(10) Pub. No.: US 2021/0228804 A1
(43) Pub. Date: Jul. 29, 2021

(54) MEAL INSULIN DETERMINATION FOR IMPROVED POST PRANDIAL RESPONSE

(71) Applicant: Insulet Corporation, Acton, MA (US)

(72) Inventors: Eric BENJAMIN, Cambridge, MA (US); Joon Bok LEE, Acton, MA (US); Yibin ZHENG, Hartland, WI (US); Jason O'CONNOR, Acton, MA (US); Ashutosh ZADE, San Diego, CA (US)

(21) Appl. No.: 17/150,461

(22) Filed: Jan. 15, 2021

Related U.S. Application Data
(60) Provisional application No. 62/965,136, filed on Jan. 23, 2020.

(51) Int. Cl. A61M 5/172; A61B 5/145; A61K 38/28

(52) U.S. Cl. CPC A61M 5/1723; A61M 2005/14208; A61K 38/28; A61B 5/14532

(57) ABSTRACT

Disclosed are a device, system, methods and computer-readable medium products that provide techniques to implement functionality to receive information related to a meal and delivery of a meal bolus. The received information may be stored and utilized in the generation of insulin delivery profiles. An estimate a mealtime insulin need based on the generated insulin delivery profiles may be generated. A dose of insulin may be determined that satisfies an estimated mealtime insulin need based on the generated insulin delivery profiles. The determined dose of insulin may be output at a time corresponding to a mealtime.

### CROSS-REFERENCE TO RELATED APPLICATIONS

This application claims the benefit of the filing date of U.S. Provisional Application Ser. No. 62/965,136, filed Jan. 23, 2020, the entire contents of which are incorporated herein by reference in their entirety.

### BACKGROUND

Due to the complicated and dynamic nature of the human body's response to insulin patients may end up in a hypoglycemic or hyperglycemic state after being treated with insulin therapy. This outcome is undesirable for many reasons: hypoglycemia creates an immediate risk of a severe medical event (seizure, coma, death) while hyperglycemia creates long term negative health effects as well as the risk of ketoacidosis. Whether a person ends up in one of these states depends on a very complicated combination of many factors and sources of error. One source of error comes from the assumed duration of insulin action in the patient. Insulin therapy systems use a value, or function, to determine how long insulin remains active in a patient.

The state of the art of meal bolusing is users of insulin pumps estimating the carbohydrate content of each meal, estimating their insulin-to-carbohydrate clinical parameter, and converting the carbohydrate content of the meal into an insulin dose.

There are as of yet non commercialized methods of user indications of bolusing that attempt to reduce the user's meal estimation needs by allowing them to indicate a small/medium/high carbohydrate content, but these implementations do not consider past insulin delivery history, and instead utilize known distributions of meal carbohydrate (i.e., carbon-hydrogen-oxygen (CHO)) content to estimate what a small/medium/high meal content would be.

Compensation of post prandial meal glucose excursions is one of the key challenges in glucose control for any insulin delivery system. Automated insulin delivery systems have been demonstrated to perform excellent glucose control during periods without significant meal disturbances; however, their performances during post prandial periods generally are suboptimal and require user guided bolusing for safe glycemic control. Due to the need to estimate the carbohydrate content, timing of meal ingestion, and life events surrounding this meal, such user guided bolusing is a significant source of increased risk of hypoglycemia or hyperglycemia to the user.

### BRIEF DESCRIPTION OF THE DRAWINGS

FIG. 1 illustrates an example of a process for providing an estimate of a mealtime insulin need.

FIG. 2 shows a flow chart of an example of a process that utilizes a user's total daily insulin needs to generate an estimate of a user's mealtime insulin needs.

FIG. 3 illustrates an example of a subprocess may be implemented to determine a meal bolus to be delivered.

FIG. 4A illustrates an example of a user interface for indicating ingestion of a meal.

FIG. 4B illustrates another example of a user interface for indicating ingestion of a meal.

FIG. 5 illustrates a functional block diagram of drug delivery system suitable for implementing the example processes and techniques described herein.

### DETAILED DESCRIPTION

The disclosed examples provide an estimate insulin dosage to meet a user's mealtime insulin needs based on meal-related inputs received by a personal diabetes management-related algorithm from various sources.

An example may identify the average insulin delivery needs per each meal event as a proportion of a subject's total daily insulin (TDI). A meal event may be considered ingestion of carbohydrates during a specific commonly-named meal, such as breakfast, lunch, and dinner, as well as snacks at particular times, such as a mid-afternoon snack, a bedtime snack, an after-exercise recovery snack, or the like. For example, the timing or reference to the snack and the meal may be customized for the particular user.

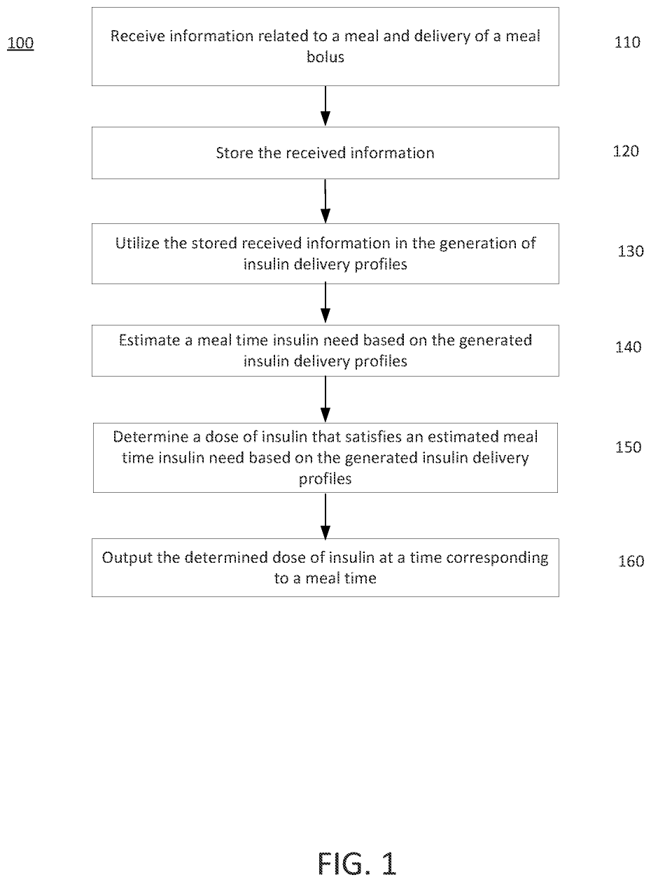

FIG. 1 illustrates an example of a process for providing an estimate of a mealtime insulin need. In the example of FIG. 1, the process 100 may be used and the average insulin delivery needs per meal event may be identified through past automated insulin delivery profiles. In an example process 100, the past automated insulin deliver profiles may be generated, for example, prior to, during and after meal events, rather than requiring the user to determine the exact quantity of their boluses through estimation of each meal's carbohydrate content and their insulin-to-carbohydrate ratios.

At 110, the algorithm may receive information related to a meal and a delivery of a meal bolus. For example, the algorithm may receive a request (e.g., via an input to a user interface) for a meal bolus (or a presentation of an amount of a bolus dose) of a personal diabetes management device or a medical device (both shown in another example) that includes, or is associated with, a time stamp or the like). The received request may indicate that a user desires to deliver a bolus in response to a meal event. The algorithm may, for example, obtain a previously received blood glucose measurement that is closest in time to the time stamp associated with the received request for a meal bolus.

At 120, the received information may be stored in a memory communicatively coupled to the algorithm. For example, the algorithm may be operable to store meal-related information, such as an estimated number of carbohydrates ingested, a time at which the meal was ingested, an amount of insulin in a meal bolus, a delivery time of the meal bolus, a blood glucose measurement value from a blood glucose measurement made closest in time to the ingestion of the mealtime or the delivery of the meal bolus, or the like. The received meal-related information may be stored for several weeks or months enabling analysis or evaluation of the received information so information, such as an average number of meals per day, the times of each of the respective meals are ingested per day, an amount of estimated carbohydrates per meal, an average blood glucose measurement value in close proximity in time to the ingestion of each meal, average blood glucose measurement values at regular increments of time approximately after the ingestion of meals (e.g., approximately 1 hour after, approximately 90 minutes after, approximately 2 hours after, approximately 3 hours after, or the like), insulin onboard and/or total daily insulin (TDI), meal-related trends, or the like, may be determined.

At 130, the algorithm may generate insulin delivery profiles utilizing the stored, received information and any additional information (e.g., insulin onboard or TDI) determined using the stored, received information. In an example of the generation of an automated insulin delivery profile, an automated insulin delivery algorithm or an artificial pancreas application, when executed by a processor, may be operable to estimate the insulin needs of a user for each meal using a number (e.g., at least one or more) of samples in insulin delivery profiles that are collected prior to, during and after the meal events. The estimated insulin needs of each meal may be coordinated with the user's total insulin needs per day to account for changes in insulin needs over time and allow the user to indicate their ingestion pattern instead of attempting to calculate exact insulin needs for each meal and input the calculated insulin needs into the algorithm or AP application.

At 140, the algorithm may estimate a mealtime insulin need based on the generated insulin delivery profiles. In a specific example, the insulin delivery profile for a dinner meal may indicate that the user provides an average meal bolus dose of 8 units (U) of insulin prior to ingesting a meal, and that the user's blood glucose measurement value immediately prior to ingesting the meal is, on average, 120 mg/dL and that the user's blood glucose measurement approximately 90 minutes after ingestion of the meal is 180 mg/dL. Of course, the values used in the specific example may be different for different users. Using this information as well as information known about a user's insulin sensitivity and the like, the algorithm may, at 150 determine a dose of insulin that satisfies an estimated mealtime insulin need based on the generated insulin delivery profiles.

At 160, the algorithm may output the estimated mealtime insulin need at a time corresponding to a mealtime. For example, the output may be in the form of an instruction and may be provided to a drug delivery device to deliver the determined dose, may be a command to generate a prompt on a display device of a personal diabetes management device, or both. The prompt may, for example, be a presentation of the determined dose or several different doses within a predetermined range of the determined dose. Alternatively, the instruction or the command may be based on a mealtime for which the determined dose is to be delivered. For example, the determined mealtime may be 5 am-10 am, which corresponds to breakfast, 11 am-2 pm, which may correspond to lunch, 2 pm-4 pm may correspond to mid-day snack, and dinner may be within the hours of 5 pm-7 pm, and Bpm-12 pm may correspond to an evening snack, or the like.

At least one outcome of executing the automated insulin delivery algorithm or an artificial pancreas application may be an ability to estimate a “starting” average insulin need per subject that may be delivered to the subject while an automated insulin delivery algorithm is active while reducing risk to the user. Another outcome of executing the automated insulin delivery algorithm or an artificial pancreas application may be an ability to estimate a range of insulin needs for different ingested meals over time through known insulin delivery profiles.

An advantage of the above outcomes may be a dramatic simplification of the meal dosing experience for users and the potential for safely eliminating a requirement for users to calculate an accurate estimate of the carbohydrate (CHO) content of each ingested meal as well as having to accurately estimate their insulin-to-carbohydrate clinical parameters.

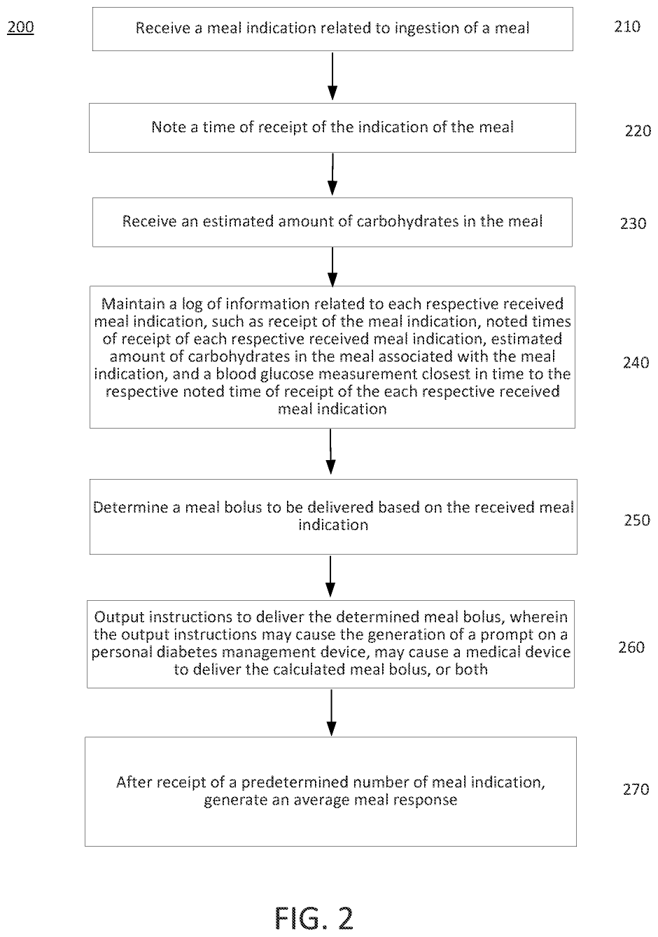

FIG. 2 shows a flow chart of an example of a process that utilizes a user's total daily insulin needs to generate an estimate of a user's mealtime insulin needs. The process 200 may be implemented using an algorithm or an AP application. At step 210, the algorithm executed by the AID system or an artificial pancreas (AP) application may receive a meal indication related to ingestion of a meal. The received indication may be by a user interaction with a meal button (e.g., a physical button) on a medical device or a user interface of a personal diabetes management device or a smartphone (e.g., a physical button or a “soft button” presented on a graphical user interface presented on a touchscreen). A time of receipt of the meal indication may be noted by the algorithm or AP application. For example, upon receipt of the indication of the meal, the algorithm or AP application may obtain a time of day and date from a clock executed by a processor, from GPS signals or the like (220). The algorithm or AP application may receive an estimated amount of carbohydrates in the meal indicated by the user (230). The received meal indication and time of receipt of the meal indication may be maintained in a log stored in a memory by the algorithm or the AP application. For example, the algorithm or the AP application may maintain a log of information related to each respective received meal indication (240). Examples of the received meal indication may include receipt of the meal indication, noted times of receipt of each respective received meal indication, estimated amount of carbohydrates in the meal associated with the meal indication, and a blood glucose measurement closest in time to the respective noted time of receipt of each respective received meal indication. At 250, the algorithm or AP application may determine a meal bolus to be delivered based on the received meal indication, wherein the determined meal bolus may be calculated by the algorithm or received via a user interface. The algorithm or AP application may, at 260, output instructions to deliver the determined meal bolus, wherein the output instructions may cause the generation of a prompt on a personal diabetes management device, may cause a medical device to deliver the calculated meal bolus, or both.

After receipt of a predetermined number of meal indications, the algorithm or AP application may generate an average meal insulin response, wherein the predetermined number of meal indications may be nine or the like and the average meal insulin response is an estimated average meal bolus for delivery after each meal (270).

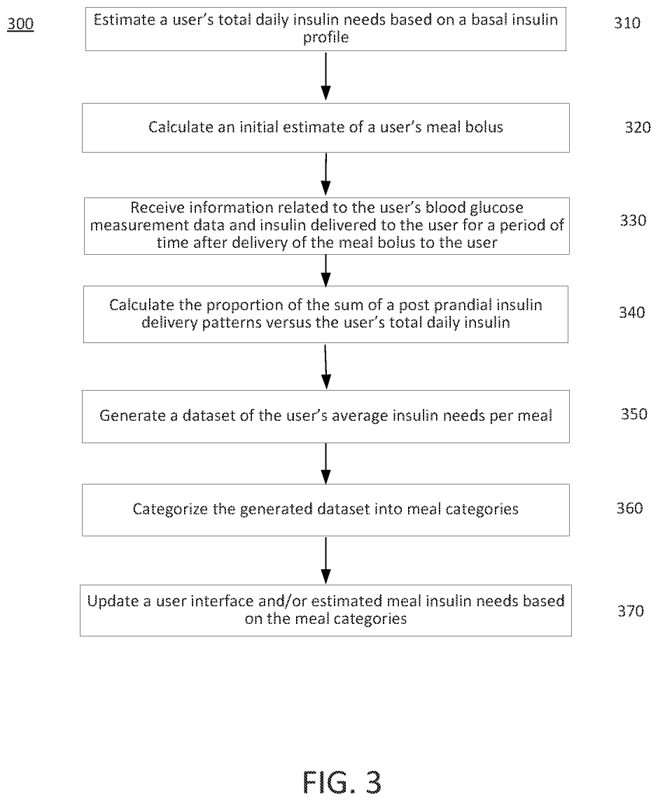

FIG. 3 illustrates an example of a subprocess may be implemented to determine a meal bolus to be delivered.

In the example of FIG. 3, an initial estimate of a user's total daily insulin (TDI) needs may be estimated by assessing the user's basal profile (e.g., the amount of insulin delivered via basal dosages throughout a 24, 48, or 72 hour time frame). In the example, the algorithm or AP application may implement a process, such as process 300, to determine an estimate of a user's total daily insulin needs based on a basal insulin profile. For example, the initial estimate of a user's total daily insulin needs may be made, at 310, according to the following example of a function:

$${TDI}_{1} = \frac{{\sum }_{j = 1}^{N} {B}_{N} \cdot T}{24}$$

where TDI₁ indicates the estimated TDI of the user for the first pod, and T indicates the number of hours of the user's basal needs covered by the N^{th} basal segment (B) in a day, represented by the 24 in the denominator. The initial estimate of TDI₁ based on the user's basal profile may be utilized for a first pod in which the algorithm and the automatic insulin delivery system are initialized to provide a mealtime insulin estimate.

In the example of FIG. 3, using the estimate of TDI₁ made at 310, the algorithm or AP application may calculate an estimate of the user's average insulin needs for each meal (320). The calculation of the user's average insulin needs may consider that the calculated TDI incorporates the sum of all the user's insulin needs, including both their basal and bolus insulin deliveries. For example, 50% of TDI₁ may be estimated to constitute the sum of insulin needs for all meal events, where the factor 50% accounts for the underlying assumption that half of the user's TDI needs correspond to the user's basal requirements and the other half accounts for the user's meal bolus requirements. This proportion may, for example, vary widely, such as 25% or 75%, depending on the user's living patterns (for example, increased activity level may mean lower basal requirements in proportion to TDI or the like) and meal patterns (for example, the ingestion of smaller meals, or partaking in fewer meals, may need higher basal requirements in proportion to TDI or the like).

In a specific example, a user's total daily insulin may be considered to contain 3 meal events per day (e.g., breakfast, lunch and dinner), meaning that an initial estimate of the meal coverage needs (i.e., a meal bolus dosage) may be set as approximately ⅓^{rd} of the sum of all meal insulin needs (i.e. total bolus dosage amounts averaged over 3 meals), or approximately ⅙^{th} of the user's TDI (i.e., the insulin delivered by the meal boluses accounts for approximately 4 hours out of the 24 hour period covered by the TDI).

In an example of open-loop use in which a user injects himself or herself and delivers insulin, the estimated dose (i.e., amount) of insulin recommended for delivery by a personal diabetes management device executing the algorithm may be constrained to limit an estimated mealtime dose of insulin to no greater than ⅙^{th} of the user's TDI.

Alternatively, due to the large amount of uncertainty in both the initial TDI estimate and the assumption of the meal bolus coverage of the TDI and the number of meals, another approach may be implemented in which approximately 50% of this initial TDI is used to estimate quantity of insulin for the meal bolus administered for each meal. Such an alternative approach of calculating an estimated meal bolus may, for example, be:

M_{est,1}=½·⅓·½·TDI₁≈0.08·TDI₁

where M_{est,1} is the first estimated meal bolus, the first occurrence of the factor ½ represents the approximately 50% of all insulin constitutes the needs for all meal events, the ⅓ factor represents the approximately 33% of the initial TDI estimated quantity assuming 3 meal boluses are delivered per day, and the second ½ represents a 50% reduction in the estimated meal bolus to compensate for the uncertainties inherent in the initial TDI estimate and meal bolus coverage assumptions. Of course, other factors may be used for different users based on the different user's physiology, e.g., metabolic rate, insulin sensitivity, and the like.

The accuracy of the initial estimated mealtime insulin needs (i.e., M_{est,1}) may be based on: 1) the possibility of the user indicating meal events for small snacks, and 2) consideration of the peak time of a meal bolus effect as 90 minutes within a total meal IOB duration of 3 hours—the amount of insulin in the bolus based on M_{est,1} may be considered to cover the insulin required by the user to compensate for the amount of insulin required during the initial peak insulin action under standard meal bolusing.

In addition, due to utilizing an estimated mealtime bolus M_{est,n}, an automated insulin delivery algorithm may be enabled to suspend delivery of basal insulin for approximately 1.5 hours (i.e. approximately 90 minutes), which results in 1.5· 1/48≈3% of possible insulin deficiency accumulation (where the 48 factor corresponds to 50% of the user's TDI accounting for the user's basal needs, which is then converted as an hourly rate per day, or ½· 1/48). The suspension of basal insulin delivery for the approximately 1.5 hours may compensate for a proportion of any excess insulin delivered above basal that may be caused by the delivery of the estimated meal bolus. This enables the system to compensate for a discrepancy in the meal estimates by an amount equal to suspension of basal delivery by approximately 1.5 hours, reducing the severity of adverse impacts to the user's blood glucose measurements due to the discrepancy.

In this example when determining a meal bolus, such as in step 250 of FIG. 2, an element of adaptivity is preserved by assessing the insulin needs per meal as proportions of the user's TDI instead of actual units of insulin. For example, a meal estimate may be recalculated after each meal button interaction by a user, thus converging over time to a value that represents the user's true meal insulin needs for each meal based on the button interaction corresponding to the respective meal.

In alternate examples, the meal event indicators may be disabled, or their responses may be muted for multiple interactions of the system within a short period of time, such as 90 minutes, 105 minutes, or the like, to match an average peak time of insulin action of a user. As a result, the AP algorithm or application can compensate for the risk of users accidentally activating the meal indicator in situations where the users do not desire an event, or mistakenly re-activating the meal indicator during occurrences of hyperglycemia following a meal when the users had already activated the indicator once for this meal.

Returning to the example of FIG. 3, the AP algorithm or application may receive information related to the user's blood glucose measurement data and insulin delivered to the user for a period of time after delivery of the meal bolus to the user (330).

For example, after a certain number of meals (e.g. five to ten meals), the average meal insulin response may be determined based on the closed-loop delivery of insulin. Using the received information, the algorithm or AP application may, at 340, calculate the proportion of the sum of the 5 hour post prandial insulin delivery patterns versus the user's TDI:

$${M}_{1 … N , i} = [ \begin{matrix}{\sum }_{j = 1}^{60} \frac{I ( {T}_{M 1} + j )}{{TDI}_{1}} & {\sum }_{j = 1}^{60} \frac{I ( {T}_{M 2} + j )}{{TDI}_{1}} & {\sum }_{j = 1}^{60} \frac{I ( {T}_{M 3} + j )}{{TDI}_{1}} & … & {\sum }_{j = 1}^{60} \frac{I ( {T}_{M 4} + j )}{{TDI}_{1}}\end{matrix} ]$$

Where T_{Mn} represents the time of the user's meal interaction, and I(t) represents the insulin delivery during the t-th sample of the time of the user's meal interaction, MN represents a user's mealtime insulin need for the N^{th} meal, and j represents the number of 5-minute measurements of blood glucose by a continuous glucose monitor (shown in another example) or the like that may have passed since the initiation of the user's interactions with the meal button. In this example, the 60 in the summation represents 5 hours of samples. The 5 hours is equivalent to a common post prandial period. Of course, the 5 hours may be modified depending upon the physiology of the user, and may be 3 hours, 4 hours, 5 hours, 6 hours, or the like.

Alternatively, at 340, the total insulin delivery for the user may be calculated as the sum of all insulin deliveries during the defined post prandial period and any further excessive insulin delivery or lack of insulin delivery based on the glucose outcomes following this post prandial period. The user's glucose concentration and Insulin-On-Board at the end of each post prandial period can be translated to the user's expected final glucose concentration, and the difference between this expected final glucose concentration and the user's target glucose concentration can be assessed to determine whether the total insulin delivery during this period was excessive, lacking, or matching the user's actual needs. This can be captured as the following:

$${M}_{new 1 … N , i} = [ \begin{matrix}{\sum }_{j = 1}^{60} \frac{I ( {T}_{M 1} + j )}{{TDI}_{1}} + Ma {dj}_{1} & {\sum }_{j = 1}^{60} \frac{I ( {T}_{M 2} + j )}{{TDI}_{1}} - Ma {dj}_{2} & … & {\sum }_{j = 1}^{60} \frac{I ( {T}_{M 4} + j )}{{TDI}_{1}} - Ma {dj}_{1}\end{matrix} ]$$

$${Madj}_{i} = \frac{\frac{G ( {T}_{M i} + 60 ) - target ( {T}_{M i} + 60 )}{1800 / {TDI}_{1}} - IOB ( G ( {T}_{M i} + 60 ) )}{{TDI}_{1}}$$

where the second term, i.e., Madjᵢ, in each element of the vector of meal insulin needs, i.e. M_{new1 . . . N,i} represents the component for IOB and glucose compensation. Here, target(i) and G(i) represent the user's glucose and glucose target at the i^{th} time, and 1800/TDI is a representation of the user's correction factor. This part of the term translates the user's deviation from the user's final glucose concentration to the target into insulin needs (excess or lack thereof). IOB(i) represents the user's residual insulin action at the i^{th} time, or the end of the period of interest, as this residual IOB did not take action in the user's current glucose concentration and should thus be discounted.

At 350, the calculated proportions may be used to generate a dataset of the user's average insulin needs per meal in terms of proportion of TDI. In a further example, the individual sum of insulin deliveries following the 5-hour post prandial period after each meal button interaction can be separately stored as M_{1 . . . N}. As a result, a more accurate estimate for insulin delivery at time of interaction (e.g., receipt of a meal indication or meal bolus request) with the next pod, M_{est,2} may be calculated by taking an average of these meal events:

$${M}_{est , 2} = \frac{{\sum }_{i = 1}^{N} {M}_{i}}{N}$$

Where M_{est,2} is an estimated mealtime insulin need subsequent to the initial estimate M_{est,1}.

In 360, the dataset of post prandial meals may be categorized by the algorithm or the AP application into multiple sets of different meals. For example, the distribution of these generated dataset may be categorized into meal categories of “snacks”, “small meals,” “medium meals,” and “large meals.” For example, this may be executed by calculating four equally distributed percentiles, where the 25th, 50th, 75th, and 100th percentiles that may correspond to the respective meal categories that may be calculated as:

$$T {H}_{25 … 100} = 100 \frac{( 25 … 100 ) + 0.5}{N} th datapoint$$

the post prandial meal insulin needs that correspond to the 25^{th}, 50^{th}, 75^{th}, and 100^{th} percentiles may be identified as TH₂₅, TH₅₀, TH₇₅, and TH₁₀₀, respectively. Linear interpolations may be utilized to compute percentiles when there are an insufficient number of datapoints available. The user's meal insulin needs between each respect TH value may be considered to correspond to a respective meal category of “snack” (i.e., 25^{th} percentile), “small meal” (i.e., 50^{th} percentile), “medium meal” (i.e., 75^{th} percentile), and “large meal” (i.e., 100^{th} percentile), and averages of meal insulin needs of each categories can be defined as the meal insulin needs Mesa, Mₑₛₜ₂, Mₑₛₜ₃, and Mₑₛₜ₄. Based on the meal categories, the algorithm or AP application may update a user interface on a medical device, or a personal diabetes management device and/or estimated meal insulin needs for each respective meal category (370).

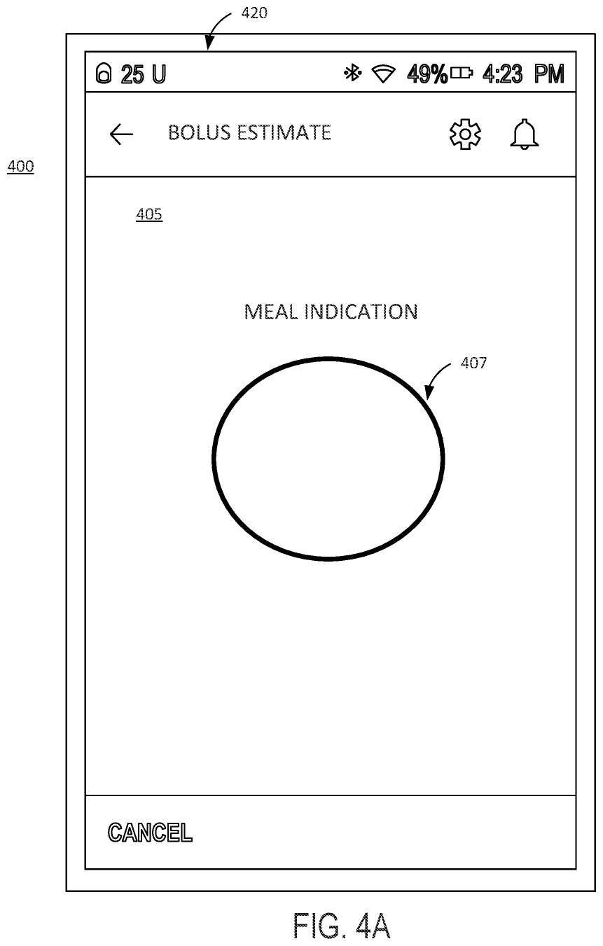

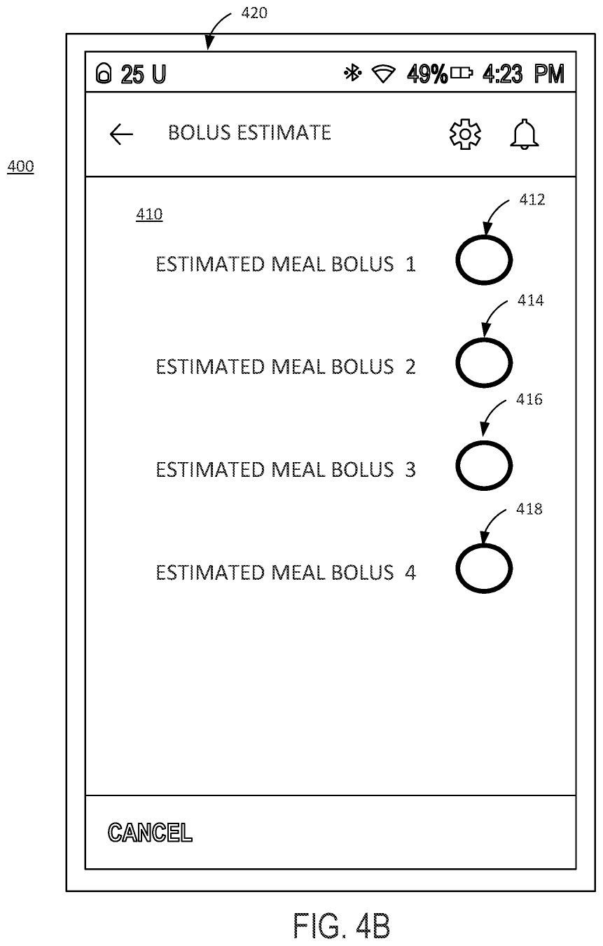

FIGS. 4A and 4B illustrate examples of user interfaces for indicating ingestion of a meal. In the example of FIG. 4A, a user may interact with a single “meal event” indicator, while a closed-loop, automated insulin delivery algorithm is active during a first use (with respect to the disclosed examples) of the system

As described with reference to step 210 of FIG. 2 above, a user may indicate the ingestion, or imminent ingestion, of a meal by actuating a meal indication button. The example of FIG. 4A illustrates a personal diabetes management device 400 on which is presented a user interface 405 for indicating ingestion of a meal. The user interface 405 may include a “soft” button 407 that the user may interact with to indicate that a meal is about to be ingested or has been ingested. While the examples of FIGS. 4A and 4b show “soft buttons,” the user interface may also be, or may include, a microphone, “hard buttons”, keypad or the like. In the example of a microphone user interface, the AP application or algorithm may have access to natural language recognition software that enables the input of voice commands, such as “eating a meal” or the like.

In response to the user interaction with the “meal event” indicator, such as 407 of FIG. 4A, the closed-loop, automated insulin delivery algorithm may be operable to cause a dose of insulin approximately equivalent to the estimated meal bolus (M_{est,1}) to be delivered to the user. In a further example, in response to actuation of the meal event indicator 407, the AP application or algorithm may relax constraints within the algorithm (such as the insulin-on-board (IOB) constraint, maximum total daily insulin limit, or the like) as executed either by the AP application or separately from the AP application to allow the algorithm to compensate for the remaining 50% of the estimated average meal insulin needs for each event. The algorithm may operate separately from the AP application and provide information to the AP application related to the meal bolus calculation or may even provide the amount of insulin that is to be provided by as meal bolus. The meal bolus may be delivered via a wearable drug delivery device, by a syringe, a “smart pen” drug delivery device, or the like.

In the example of FIG. 4B, a user may interact with several “meal event” indicators, while a closed-loop, automated insulin delivery algorithm is active. Users' interactions with these meal buttons allow the system to categorize the resulting closed loop insulin delivery patterns over time by assessing the insulin delivery that occurs following each button interaction instance.

In an aspect discussed further with reference to FIG. 4B, a user may interact with several “meal event” indicators that enable an accumulation of data that may be used to make more precise estimations of a user's meal insulin needs. For example, users may indicate to the algorithm or the AP application that a meal is about to be ingested or has been ingested via an interaction with a user interface either on a medical device (operable to deliver insulin) or a personal diabetes management device (both shown in another example). In an example, the personal diabetes management device or other computing device, such as a smart phone, smart watch, fitness device, or other wearable accessory, may be operable to provide a user interface that presents meal event indicators. Alternatively, or in addition, a pod or medical device (shown in another example) may have a user interface (described with reference to another example) that enables a user to interact with the pod or medical device and deliver the estimated mealtime insulin need.

For example, a user interface may be presented with a single button labeled “meal ingested” or the like, whenever a user has a meal, the user presses the button. The system takes the sum of all insulin delivered for the post prandial period, such as for the next 4, 5 or 6 hours, or the like. The automated insulin delivery system will be delivering more than basal for the time period during which the user's blood glucose measurement values are high (e.g., greater than 120 mg/dL). After a certain amount of time has passed, the AID system determines that at each interaction of the meal button, the system has to deliver 2 units of insulin half of the time, while in another instance, the system has to deliver 4 units of insulin a quarter of the time, and the last quarter, the system has to deliver 6 units. Based on this, the system may be configured to be operable to present multiple meal buttons (e.g., 3 in this example) that indicates a meal, e.g. snack, medium meal or large meal in this example. The user may have the option to select one of the three presented buttons.

It may take three pods or 9 days for the system to obtain enough data to enable generation of the multiple meal buttons by the algorithm. The number of meal boluses and correction boluses on average delivered by a user is approximately 6 total boluses. For a user, this may be approximately 54 total boluses (i.e., 6 boluses per day times 9 days).

A setting for the number of boluses may be increased or decreased for more or less stability, respectively, by the algorithm or AP application. The change in the number of boluses may, for example, depend on a measurement of the stability of the user's glucose measurements; such as a consistent % time in the 70-180 mg/dL range across multiple days, or other methods. For example, the number of boluses may be reduced from approximately 50 to approximately 25 or increased to 75 depending upon the stability of the user's blood glucose measurements or lifestyle (e.g., more active or less active or the like). The reduction in the number of boluses may result in lower accuracy and less conservative dosages of insulin being delivered. For example, instead a half (½) the number of boluses, the algorithm or AP application may reduce the number of boluses by one-quarter (¼) to be more conservative. Alternatively, the setting may be set to deliver the average of all of the values instead of developing categories, the reason for this may be, for example, insufficient data or the like.

In some examples, a user may decide that the amount of insulin delivered via the meal button feature is not sufficient. In such instances, the user may reset the insulin delivery history and other received information (e.g., during step 330 of FIG. 3) used to generate the estimated mealtime insulin.

In closed loop operation, when the blood glucose measurement goes high in response to a meal the AID system begins delivering an amount of insulin above the basal dosage. Eventually, after a period of time, the blood glucose measurement returns to approximately target blood glucose measurement. Using this amount of time and the amount of insulin delivered, the algorithm controlling the AID system may modify the amount of insulin above the basal dosage delivered to counteract the increased blood glucose measurement.

The resulting meal interaction insulin deliveries (e.g. M_{Est,1 . . . 4}) for a subsequent pod may be alternately indicated by the user through four different meal interaction buttons. For example, FIG. 4B shows a personal diabetes management device 400 that presents a user interface 410 on a display 420 of personal diabetes management device 400. Each respective estimated meal bolus 1, estimated meal bolus 2, estimated meal bolus 3, and estimated meal bolus 4 may deliver a different amount of insulin dependent upon the type of meal that triggers the need for a bolus request. For example, a user having a snack (or a “snacks” associated with TH₂₅) may actuate meal interaction button 412 associated with estimated meal bolus 1, or if the user is having breakfast (or a “small meal” associated with TH₅₀) may actuate button 414 associated with estimated meal bolus 2. Alternatively, if the user is having lunch (or a “medium meal” associated with TH₇₅), the user may actuate button 416 associated with estimated meal bolus 3, and if the user is having dinner (or a “large meal” associated with TH₁₀₀), the user may actuate button 418 associated with estimated meal bolus 4. Depending on the type of meal, the user does not have to estimate an amount of carbohydrates.

The presence of the meal interaction buttons 412, 414, 416 and 418 are advantageous because of the reduced risk of overdose for meal events having lower than average meal carbohydrates in comparison to utilizing just one meal interaction button.

In another example, cluster analysis may be conducted on each available dataset to identify groups of meal insulin deliveries that may be considered to be similar. Different forms of cluster analysis may be used. In a specific example, k-means cluster analysis may be conducted on the meal insulin delivery datasets to determine the centroids for each of the meals. For example, the k-means analysis may utilize four expected clusters as in the above percentile-based approaches (e.g., TH₂₅, TH₅₀, TH₇₅, and TH₁₀₀). In other examples, distribution or density-based clustering algorithms may be implemented to not only discover the average clusters of the meals but also the number of clusters that match the user's individualized meal ingestion patterns. This may result, for example, in a few potential meal insulin delivery options being proposed to the user for subsequent system interactions personalized to each user. For example, a default maximum number of potential meal insulin delivery options may be limited to 4 for an optimal user experience. However, in other examples, the number of potential meal insulin delivery options may be customized to include addition potential meal insulin delivery options (such as mid-night snack, afternoon snack, or the like).

The analyses described above may, for example, be repeated as greater amounts of data is gathered to better improve the accuracy of the estimate of the mealtime insulin delivery needs M_{est,1 . . . 4} of each user.

In other examples, the thresholds for different meal sizes may be set to unequal sizes, such as 10%, 40%, 70%, and 100%, or other guidelines. Alternatively, or in addition, the dataset of meal insulin delivery profiles may be implemented with a weighting factor that reduces the impact of outliers in cases where the associated glucose profiles with each meal insulin delivery profile may be abnormal or missing.

In another example, a user may have a sudden change in meal-time habits due to some event, such as, a work-related travel schedule, a holiday event, a health-related event or the like. As a result of the sudden change, the four estimated meal bolus options (via meal interaction buttons 412-418) presented in the user interface 410 may not apply to the user's new mealtimes or carbohydrate intake. If such a sudden change occurs, the algorithm or AP application may provide an optional prompt in which the user via the user interface 410 enabling a user to revert to entering estimated carbohydrates for each meal. Alternatively, or in addition, the user may indicate to the algorithm or AP application that the algorithm or AP application should disregard this particular meal information (e.g., insulin delivered during post prandial period, indicated carbohydrates, blood glucose measurements) when calculating an estimated meal insulin delivery (or meal bolus).

In the examples of FIGS. 1-4B, the example processes may be implemented by programming code, such as an AP application or an algorithm, which is executed by a processor. The AP application or algorithm when executed by a processor may utilize inputs and calculations as described with respect to the foregoing examples.

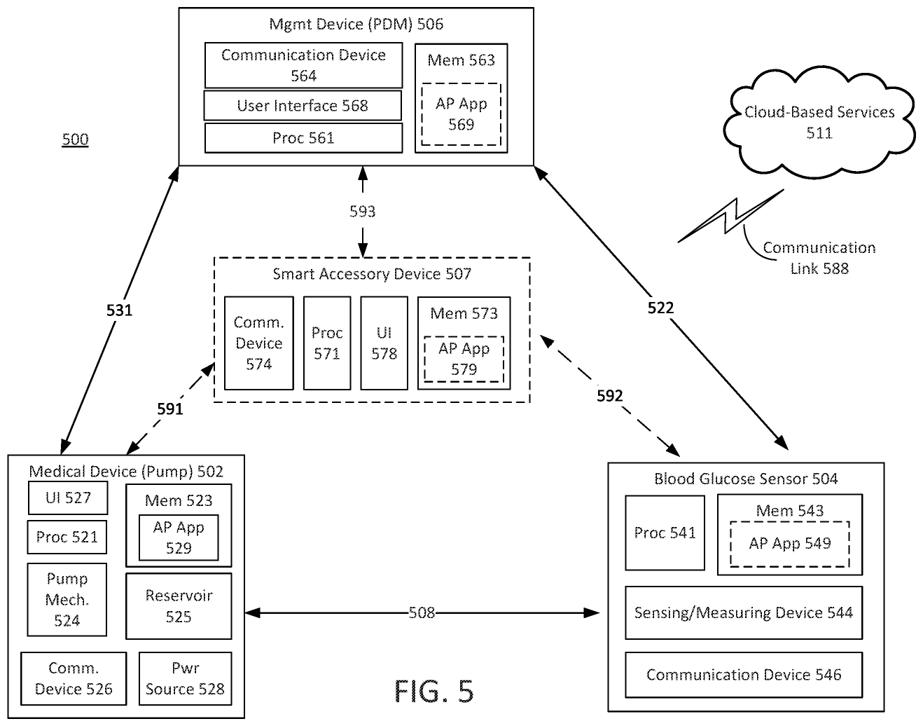

It may be helpful to discuss an example of a drug delivery system that may implement the process example of FIGS. 1-4B. FIG. 5 illustrates an example of a drug delivery system 500.

The drug delivery system 500 may be operable to implement the process examples illustrated in FIGS. 1-4B by executing an AP application or algorithm that includes functionality to determine.

The drug delivery system 500 may be an automated drug delivery system that may include a medical device (pump) 502 (also referred to as “a drug delivery device” or “a wearable drug delivery device”), a blood glucose sensor 504 (also referred to as “a continuous glucose monitor” or “a blood glucose measurement device”), and a management device (PDM) 506. The system 500, in an example, may also include a smart accessory device 507, which may be operable to communicate with the other components of system 500 either via a wired or wireless communication link, such as 591, 592 or 593.

In an example, the medical device 502 may be attached to the body of a user, such as a patient or diabetic, and may deliver any therapeutic agent, including any drug or medicine, such as insulin, morphine or the like, to the user. The medical device 502 may, for example, be a wearable device worn by the user. For example, the medical device 502 may be directly coupled to a user (e.g., directly attached to a body part and/or skin of the user via an adhesive or the like). In an example, a surface of the medical device 502 may include an adhesive (not shown) to facilitate attachment to a user.

The medical device 502 may include a few components to facilitate automated delivery of a drug (also referred to as a therapeutic agent) to the user. The medical device 502 may be operable to store the drug (i.e., insulin) and to provide the drug to the user. The medical device 502 is often referred to as a pump, or an insulin pump, in reference to the operation of expelling insulin from the reservoir 525 for delivery to the user. While the examples refer to the reservoir 525 storing insulin, the reservoir 525 may be operable to store other drugs or therapeutic agents, such as morphine or the like, that are suitable for automated delivery.

In various examples, the medical device 502 may be an automated, wearable drug delivery device. For example, the medical device 502 may include a reservoir 525 for storing the drug (such as insulin), a needle or cannula (not shown) for delivering the drug into the body of the user (which may be done subcutaneously, intraperitoneally, or intravenously), and a pump mechanism (mech.) 524, or other drive mechanism, for transferring the drug from the reservoir 525, through a needle or cannula (not shown), and into the user. The pump mechanism 524 may be fluidly coupled to reservoir 525, and communicatively coupled to the medical device processor 521. The medical device processor 521 may also enable the medical device 502 (also referred to as a drug delivery device. The medical device processor 521 may be communicatively coupled to the processor 561 of management device 506 or processor 571 of smart accessory device 507. The medical device processor 521 may be operable to receive the outputted instructions to deliver the determined meal bolus, actuate the pump mechanism in response to the received instructions, and output a signal containing information indicating an amount of insulin delivered by the pump in response to the received instruction.

The medical device 502 may also include a power source 528, such as a battery, a piezoelectric device, or the like, for supplying electrical power to the pump mechanism 524 and/or other components (such as the medical device processor 521, memory 523, and the communication device 526) of the medical device 502. Although not shown, an electrical power supply for supplying electrical power may similarly be included in each of the sensor 504, the smart accessory device 507 and the management device (PDM) 506.

The blood glucose sensor 504 may be a device communicatively coupled to the processor 561 or 521 and may be operable to measure a blood glucose value at a predetermined time interval, such as every 5 minutes, or the like. The blood glucose sensor 504 may provide several blood glucose measurement values to the AP applications operating on the respective devices (e.g., 529, 549 569, or 579).

The medical device 502 may provide the insulin stored in reservoir 525 to the user based on information (e.g., blood glucose measurement values, predicted future blood glucose measurements, evaluations based on a user request for a bolus, an user interaction with PDM 506, medical device 502, sensor 504 or smart accessory device 507), evaluations of missing blood glucose measurements and the other information provided by the sensor 504, smart accessory device 507, and/or the management device (PDM) 506. For example, the medical device 502 may contain analog and/or digital circuitry that may be implemented as a medical device processor 521 (or controller) for controlling the delivery of the drug or therapeutic agent. The circuitry used to implement the medical device processor 521 may include discrete, specialized logic and/or components, an application-specific integrated circuit, a microcontroller or processor that executes software instructions, firmware, programming instructions or programming code (enabling, for example, the artificial pancreas application (AP App) 529 as well as the process examples of FIGS. 1-4B) stored in memory 523, or any combination thereof. For example, the medical device processor 521 may execute a control algorithm, such as an artificial pancreas application 529, and other programming code that may make the medical processor 521 operable to cause the pump to deliver doses of the drug or therapeutic agent to a user at predetermined intervals or as needed to bring blood glucose measurement values to a target blood glucose value. In an example, the AP application (App) 529 may include programming code that is operable upon execution by the medical device processor 521 to provide the example processes for adjusting or modifying duration of insulin action settings, confidence values, insulin delivery settings, storing blood glucose measurement values in memory, or the like as described with reference to FIGS. 1-4B. The size and/or timing of the doses may be programmed, for example, into an artificial pancreas application 529 by the user or by a third party (such as a health care provider, medical device manufacturer, or the like) using a wired or wireless communication link, such as 531, between the medical device 502 and a management device 506 or other device, such as a computing device at a healthcare provider facility. In an example, the pump or medical device 502 is communicatively coupled to the processor 561 of the management device via the wireless communication link 531 or via a wireless communication link, such as 591 from smart accessory device 507 or 508 from the sensor 504. The pump mechanism 524 of the medical device 502 may be operable to receive an actuation signal from the processor 561, and in response to receiving a command signal or actuation signal, expel insulin from the reservoir 525 based on the evaluations and process steps performed in the process examples of FIGS. 1-4B.

In an operational example, the AP application 569 may be executing in the management device 506 and control delivery of insulin. For example, the AP application 569 may be operable to determine timing of an insulin dose and may output a command signal to the medical device 502 that actuates the pump mechanism 524 to deliver insulin dose based on the evaluations and process steps performed in the process examples of FIGS. 1-4B.

The other devices in the system 500, such as management device 506, smart accessory device 507 and sensor 504, may also be operable to perform various functions including controlling the medical device 502. For example, the management device 506 may include a communication device 564, a processor 561, and a management device memory 563. The management device memory 563 may store an instance of the AP application 569 that includes programming code, that when executed by the processor 561 provides the process examples described with reference to the examples of FIGS. 1-4B. The management device memory 563 may also store programming code for providing the process examples described with reference to the examples of FIGS. 1-4B.

The smart accessory device 507 may be, for example, an Apple Watch®, other wearable smart device, including eyeglasses, provided by other manufacturers, a global positioning system-enabled wearable, a wearable fitness device, smart clothing, or the like. Like the management device 506, the smart accessory device 507 may also be operable to perform various functions including controlling the medical device 502. For example, the smart accessory device 507 may include a communication device 574, a processor 571, and a memory 573. The memory 573 may store an instance of the AP application 579 that includes programming code for providing the process examples described with reference to the examples of FIGS. 1-3.

The memory 573 may also as store programming code and be operable to store data related to the AP application 579. The sensor 504 of system 500 may be a continuous glucose monitor (CGM) as described above, that may include a processor 541, a memory 543, a sensing or measuring device 544, and a communication device 546. The memory 543 may, for example, store an instance of an AP application 549 as well as other programming code and be operable to store data related to the AP application 549 and process examples described with reference to FIGS. 1-4B. The AP application 549 may also include programming code for providing the process examples described with reference to the examples of FIGS. 1-4B.

Instructions for determining the delivery of the drug or therapeutic agent (e.g., as a bolus dosage) to the user (e.g., the size and/or timing of any doses of the drug or therapeutic agent) may originate locally from the medical device 502 or may originate remotely and be provided to the medical device 502. In an example of a local determination of drug or therapeutic agent delivery, programming instructions, such as an instance of the artificial pancreas application 529, stored in the memory 523 that is coupled to the medical device 502 may be used to make determinations, such as those described with reference to FIGS. 1-4B by the processor 521 of the medical device 502. In addition, the medical device 502 may be operable to communicate with the cloud-based services 511 via the communication device 526 and the communication link 588.

Alternatively, the remote instructions may be provided to the medical device 502 over a wired or wireless communication link (such as 531) by the management device (PDM) 506, which has a processor 561 that executes an instance of the artificial pancreas application 569, or the smart accessory device 507 (via communication link 591), which has a processor 571 that executes an instance of the artificial pancreas application 569 as well as other programming code for controlling various devices, such as the medical device 502, smart accessory device 507 and/or sensor 504. The medical device 502 may execute any received instructions (originating internally or from the management device 506) for the delivery of the drug or therapeutic agent to the user. In this way, the delivery of the drug or therapeutic agent to a user may be automated.

In various examples, the medical device 502 may communicate via a wireless communication link 531 with the management device 506. The management device 506 may be an electronic device such as, for example, a smart phone, a tablet, a dedicated diabetes therapy management device, or the like. The management device 506 may be a wearable wireless accessory device. The wireless communication links 508, 531, 522, 591, 592 and 593 may be any type of wireless communication link provided by any known wireless standard. As an example, the wireless communication links 508, 531, 522, 591, 592 and 593 may enable communications between the medical device 502, the management device 506 and sensor 504 based on, for example, Bluetooth®, Wi-Fi®, a near-field communication standard, a cellular standard, or any other wireless optical or radio-frequency protocol.

The sensor 504 may be a glucose sensor operable to measure blood glucose and output a blood glucose value or data that is representative of a blood glucose value. For example, the sensor 504 may be a glucose monitor or a continuous glucose monitor (CGM). The sensor 504 may include a processor 541, a memory 543, a sensing or measuring device 544, and communication device 546. The communication device 546 of sensor 504 may include one or more sensing elements, an electronic transmitter, receiver, and/or transceiver for communicating with the management device 506 over a wireless communication link 522 or with medical device 502 over the link 508. The sensing or measuring device 544 may include one or more sensing elements, such as a glucose measurement, heart rate monitor, or the like. The processor 541 may include discrete, specialized logic and/or components, an application-specific integrated circuit, a microcontroller or processor that executes software instructions, firmware, programming instructions stored in memory (such as memory 543), or any combination thereof. For example, the memory 543 may store an instance of an AP application 549 that is executable by the processor 541.

Although the sensor 504 is depicted as separate from the medical device 502, in various examples, the sensor 504 and medical device 502 may be incorporated into the same unit. That is, in various examples, the sensor 504 may be a part of the medical device 502 and contained within the same housing of the medical device 502 (e.g., the sensor 504 may be positioned within or embedded within the medical device 502). Glucose monitoring data (e.g., measured blood glucose values) determined by the sensor 504 may be provided to the medical device 502, smart accessory device 507 and/or the management device 506 and may be used to perform the functions and deliver doses of insulin for automated delivery of insulin by the medical device 502 as described with reference to the examples of FIGS. 1-4B.

The sensor 504 may also be coupled to the user by, for example, adhesive or the like and may provide information or data on one or more medical conditions and/or physical attributes of the user. The information or data provided by the sensor 504 may be used to adjust drug delivery operations of the medical device 502.

In an example, the management device 506 may be a computing device operable to manage a personal diabetes treatment plan via an AP application or an algorithm. The management device 506 may be used to program or adjust operation of the medical device 502 and/or the sensor 504. The management device 506 may be any portable electronic, computing device including, for example, a dedicated controller, such as processor 561, a smartphone, or a tablet. In an example, the management device (PDM) 506 may include a processor 561, a management device memory 563, and a communication device 564. The management device 506 may contain analog and/or digital circuitry that may be implemented as a processor 561 (or controller) for executing processes to manage a user's blood glucose levels and for controlling the delivery of the drug or therapeutic agent to the user. The processor 561 may also be operable to execute programming code stored in the management device memory 563. For example, the management device memory 563 may be operable to store an artificial pancreas (AP) application 569 that may be executed by the processor 561. The processor 561 may when executing the artificial pancreas application 569 may be operable to perform various functions, such as those described with respect to the examples in FIGS. 1-3.

The communication device 564 may be a receiver, a transmitter, or a transceiver that operates according to one or more radio-frequency protocols. For example, the communication device 564 may include a cellular transceiver and a Bluetooth transceiver that enables the management device 506 to communicate with a data network via the cellular transceiver and with the sensor 504 and the medical device 502. The respective transceivers of communication device 564 may be operable to transmit signals containing information useable by or generated by the AP application or the like. The communication devices 526, 546 and 576 of respective medical device 502, sensor 504 and smart accessory device 507 may also be operable to transmit signals containing information useable by or generated by the AP application or the like.

The medical device 502 may communicate with the sensor 504 over a wireless communication link 508 and may communicate with the management device 506 over a wireless communication link 531. The sensor 504 and the management device 506 may communicate over a wireless communication link 522. The smart accessory device 507, when present, may communicate with the medical device 502, the sensor 504 and the management device 506 over wireless communication links 591, 592 and 593, respectively. The wireless communication links 508, 531, 522, 591, 592 and 593 may be any type of wireless communication link operating using known wireless standards or proprietary standards. As an example, the wireless communication links 508, 531, 522, 591, 592 and 593 may provide communication links based on Bluetooth®, Wi-Fi, a near-field communication standard, a cellular standard, or any other wireless protocol via the respective communication devices 526, 546 and 564. Alternatively, one or more of the wireless communication links 508, 531, 522, 591, 592 and 593 may be wired communication links. In some examples, the medical device 502 and/or the management device 506 may include a user interface 527, 578 and 568, respectively, such as a keypad, a touchscreen display, levers, buttons, a microphone, a speaker, a display, or the like, that is operable to allow a user to enter information and allow the management device to output information for presentation to the user. For example, user interface 527 on the medical device 502 may be a series of buttons, switches, levers or the like that when actuated deliver an estimated meal bolus 1, an estimated meal bolus 3, an estimated meal bolus 3, an estimated meal bolus 4, or the like. Alternatively, or in addition, the user interface 527 may include buttons, switches, levers or the like that when actuated indicate the user has recently finished ingesting, is ingesting or about to ingest a meal.

In various examples, the drug delivery system 500 may implement the algorithm artificial pancreas (AP) application (and/or provide AP functionality) to govern or control automated delivery of insulin to a user (e.g., to maintain euglycemia—a normal level of glucose in the blood). The AP application may be implemented by the medical device 502 and/or the sensor 504. The AP application may be used to determine the times and dosages of insulin delivery. In various examples, the AP application may determine the times and dosages for delivery based on information known about the user, such as the user's sex, age, weight, or height, and/or on information gathered about a physical attribute or condition of the user (e.g., from the sensor 504). For example, the AP application may determine an appropriate amount of insulin to be delivered based on glucose level monitoring of the user through the sensor 504. The AP application may also allow the user to adjust insulin delivery. For example, the AP application may allow the user to issue (e.g., via an input) commands to the medical device 502, such as a command to request and/or deliver an insulin bolus. In some examples, different functions of the AP application may be distributed among two or more of the management device 506, the medical device (pump) 502 or the sensor 504. In other examples, the different functions of the AP application may be performed by one device, such the management device 506, the medical device (pump) 502 or the sensor 504.

As described herein, the drug delivery system 500 or any component thereof, such as the medical device may be considered to provide AP functionality or to implement an AP application. Accordingly, references to the AP application (e.g., functionality, operations, or capabilities thereof) are made for convenience and may refer to and/or include operations and/or functionalities of the drug delivery system 500 or any constituent component thereof (e.g., the medical device 502 and/or the management device 506). The drug delivery system 500—for example, as an insulin delivery system implementing an AP application—may be considered to be a drug delivery system or an AP application-based delivery system that uses sensor inputs (e.g., data collected by the sensor 504).

In an example, one or more of the devices, 502, 504, 506 or 507 may be operable to communicate via a wireless communication link 588 with cloud-based services 511. The cloud-based services 511 may utilize servers and data storage (not shown). The communication link 588 may be a cellular link, a Wi-Fi link, a Bluetooth link, or a combination thereof, that is established between the respective devices 502, 504, 506 or 507 of system 500. The data storage provided by the cloud-based services 511 may store anonymized data, such as user weight, blood glucose measurements, age, meal carbohydrate information, amount of insulin delivered by both basal and bolus, or the like. In addition, the cloud-based services 511 may process the anonymized data from multiple users to provide generalized information related to the various parameters used by the AP application. For example, an age-based general target blood glucose value may be derived from the anonymized data, which may be helpful when a user first begins using a system such as 500. The cloud-based services 511 may also provide processing services for the system 500, such as performing the process 100 in the example of FIG. 2 or additional processes, such as that described with reference to FIG. 3.

In an example, the device 502 includes a communication device 564, which as described above may be a receiver, a transmitter, or a transceiver that operates according to one or more radio-frequency protocols, such as Bluetooth, Wi-Fi, a near-field communication standard, a cellular standard, that may enable the respective device to communicate with the cloud-based services 511. For example, outputs from the sensor 504 or the medical device (pump) 502 may be transmitted to the cloud-based services 511 for storage or processing via the transceivers of communication device 564. Similarly, medical device 502, management device 506 and sensor 504 may be operable to communicate with the cloud-based services 511 via the communication link 588.

In an example, the respective receiver or transceiver of each respective device, 502, 506 or 507, may be operable to receive signals containing respective blood glucose measurement values of the number of blood glucose measurement values that may be transmitted by the sensor 504. The respective processor of each respective device 502, 506 or 507 may be operable to store each of the respective blood glucose measurement values in a respective memory, such as 523, 563 or 573. The respective blood glucose measurement values may be stored as data related to the artificial pancreas algorithm, such as 529, 549, 569 or 579. In a further example, the AP application operating on any of the management device 506, the smart accessory device 507, or sensor 504 may be operable to transmit, via a transceiver implemented by a respective communication device, 564, 574, 546, a control signal for receipt by a medical device. In the example, the control signal may indicate an amount of insulin to be expelled by the medical device 502.

Various operational scenarios and examples of processes performed by the system 500 are described herein. For example, the system 500 may be operable to implement the process examples of FIG. 1-4B.

The techniques described herein for providing functionality to receive a meal indication related to ingestion of a meal and note a time of receipt of the indication of the meal may be executed by the system 500 and the respective devices 502, 504, 506 and 507, individually or in combination as described herein. In an example, an estimated amount of carbohydrates in the meal may be received by the processor 521 medical device 502. A log of information related to each respective received meal indication may be maintained in the memory 532. A meal bolus to be delivered may be determined based on the received meal indication. The determined meal bolus may be calculated by an algorithm as described with respect to one or more of FIGS. 1-4B may be executed by the processor 521 or received via a user interface. Instructions may be output to deliver the determined meal bolus. After receipt of a predetermined number of meal indications, an average meal insulin response may be generated.

For example, the system 500 or any component thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively.

The foregoing description of example examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A non-transitory computer readable medium embodied with programming code executable by a processor, and the processor when executing the programming code is operable to perform functions, including functions to:
receive meal-related information related to a meal and delivery of a meal bolus;
store the received meal-related information;
utilize the stored received meal-related information to generate insulin delivery profiles;
estimate a mealtime insulin need based on the generated insulin delivery profiles;
determine a dose of insulin for an estimated mealtime insulin need based on the generated insulin delivery profiles; and
output the determined dose of insulin at a time corresponding to a mealtime.

2. The non-transitory computer readable medium of claim 1, wherein the non-transitory computer readable medium is further embodied with programming code executable by a processor, and the processor when executing the programming code is operable to perform functions, including functions to:
receive a request for a meal bolus, wherein the received request includes a time stamp; and
obtain a previously received blood glucose measurement that is closest in time to the time stamp associated with the received request for a meal bolus.

3. The non-transitory computer readable medium of claim 1, wherein the stored received meal-related information includes an estimated number of carbohydrates ingested, a time at which the meal was ingested, an amount of insulin in a meal bolus, a delivery time of the meal bolus, or a blood glucose measurement value from a blood glucose measurement made closest in time to the ingestion of the mealtime or the delivery of the meal bolus.

4. The non-transitory computer readable medium of claim 1, wherein the non-transitory computer readable medium is further embodied with programming code executable by a processor, and the processor when executing the programming code is operable to perform functions, including functions to:
estimate the mealtime insulin need of a user for each meal using a plurality of samples in insulin delivery profiles that are collected prior to, during and after meal events, wherein the estimated mealtime insulin need of each meal is coordinated with a user's total insulin needs per day to account for changes in the user's total insulin needs per day over time.

5. The non-transitory computer readable medium of claim 1, wherein the output is provided to a drug delivery device as an instruction to deliver the determined dose, a command to generate a prompt on a display device of a personal diabetes management device, or both.

6. A non-transitory computer readable medium embodied with programming code executable by a processor, and the processor when executing the programming code is operable to perform functions, including functions to:
receive a meal indication related to ingestion of a meal;
note a time of receipt of the indication of the meal;
receive an estimated amount of carbohydrates in the meal;
maintain a log of information related to each respective received meal indication;
determine a meal bolus to be delivered based on the received meal indication, wherein the determined meal bolus is calculated or received via a user interface;
output an instruction to deliver the determined meal bolus; and
after receipt of a predetermined number of meal indications, generate an average meal insulin response.

7. The non-transitory computer readable medium of claim 6, wherein the received meal indication includes receipt of the meal indication, a noted time of receipt of each respective received meal indication, estimated amount of carbohydrates in the meal associated with the meal indication, and a blood glucose measurement closest in time to the respective noted time of receipt of each respective received meal indication.

8. The non-transitory computer readable medium of claim 6, wherein the output instructions may cause the generation of a prompt on a personal diabetes management device, may cause a medical device to deliver the calculated meal bolus, or both.

9. The non-transitory computer readable medium of claim 6, wherein the predetermined number of meal indications may be nine or the like and the average meal insulin response is an estimated average meal bolus for delivery after each meal.

10. The non-transitory computer readable medium of claim 6, wherein the non-transitory computer readable medium is further embodied with programming code executable by a processor, and the processor when executing the programming code is operable to perform functions, including functions to:
estimate a user's total daily insulin needs based on a basal insulin profile;
calculate an initial estimate of a meal bolus for a user;
receive information related to blood glucose measurement data of the user and insulin delivered to the user for a period of time after delivery of the meal bolus to the user;
calculate a proportion of a sum of a post prandial insulin delivery patterns versus a user's total daily insulin;
generate a dataset of average insulin needs per meal;
categorize the generated dataset into meal categories; and
update a user interface and/or estimated meal insulin needs based on the meal categories.

11. The non-transitory computer readable medium of claim 6, wherein the non-transitory computer readable medium is further embodied with programming code executable by a processor, and the processor when executing the programming code is operable to perform functions, including functions to:
output instructions to deliver the determined meal bolus, wherein the instructions are based on a mealtime for which the determined meal bolus is to be delivered.

12. The non-transitory computer readable medium of claim 11, wherein the non-transitory computer readable medium is further embodied with programming code executable by a processor, and the processor when executing the programming code is operable to perform functions, including functions to:
in response to the outputted instruction, generate a prompt on a personal diabetes management device, cause a medical device to deliver the determined meal bolus, or both.

13. A device, comprising:
a processor;
a memory storing programming code, an artificial pancreas application, and operable to store data related to the artificial pancreas application, wherein the programming code and the artificial pancreas application are executable by the processor; and
a transceiver operable to receive a signal containing information usable by the artificial pancreas application and transmit a signal containing information usable by or generated by the artificial pancreas application,
wherein the processor when executing the artificial pancreas application is operable to control delivery of insulin, and to perform functions, including functions to:
receive a meal indication related to ingestion of a meal;
note a time of receipt of the indication of the meal;
receive an estimated amount of carbohydrates in the meal;
maintain a log of information related to each respective received meal indication;
determine a meal bolus to be delivered based on the received meal indication, wherein the determined meal bolus may be calculated or received via a user interface;
output an instruction to deliver the determined meal bolus; and
after receipt of a predetermined number of meal indications, generate an average meal insulin response.

14. The device of claim 13, wherein the received meal indication includes receipt of a meal indication, noted time of receipt of each respective received meal indication, estimated amount of carbohydrates in a meal associated with the meal indication, and a blood glucose measurement closest in time to a respective noted time of receipt of each respective received meal indication.

15. The device of claim 13, wherein the outputted instruction causes a prompt to be generated on a personal diabetes management device, cause a medical device to deliver the determined meal bolus, or both.

16. The device of claim 13, wherein the predetermined number of meal indications is nine and the average meal insulin response is an estimated average meal bolus for delivery after each meal.

17. The device of claim 13, wherein the processor when executing the artificial pancreas application is further operable to perform functions, including functions to:
estimate a user's total daily insulin needs based on a basal insulin profile;
calculate an initial estimate of meal bolus for a user;
receive information related to blood glucose measurement data of the user and insulin delivered to the user for a period of time after delivery of the meal bolus to the user;
calculate a proportion of a sum of a post prandial insulin delivery patterns versus a total daily insulin of the user;
generate a dataset of average insulin need per meal of the user;
categorize the generated dataset into meal categories; and
update a user interface, an estimated meal insulin need based on the meal categories, or both.

18. The device of claim 13, further comprises:
a drug delivery device communicatively coupled to the processor, wherein the drug delivery device includes a pump mechanism and a medical device processor, and the medical device processor is operable to:
receive the outputted instruction to deliver the determined meal bolus; and
actuate the pump mechanism in response to the received, outputted instruction.

19. The device of claim 18, wherein the medical device processor is further operable to:
output a signal indicating an amount of insulin delivered by the pump mechanism in response to the received, outputted instruction.

20. The device of claim 13, further comprises:
a blood glucose sensor communicatively coupled to the processor wherein the blood glucose sensor is operable to:
measure a blood glucose value at a predetermined time interval; and
provide measured blood glucose values to the processor and the artificial pancreas application.
